(19) United States
(12) Patent Application Publication
(10) Pub. No.: US 2008/0038234 A1
(43) Pub. Date: Feb. 14, 2008

(54) Bone Marrow-Related Cells Associated With Tissue Maintenance And/Or Repair

(76) Inventors: Shuji Hayashi, Nagoya-shi (JP); Makoto Inoue, Tsukuba-shi (JP); Mamoru Hasegawa, Tsukuba-shi (JP)

Correspondence Address: CLARK & ELBING LLP, 101 FEDERAL STREET, BOSTON, MA, 02110, US

(21) Appl. No.: 10/599,176

(22) PCT Filed: Mar. 22, 2005
(86) PCT No.: PCT/JP05/05144

§ 371 Date: Jun. 14, 2007

(30) Foreign Application Priority Data
Mar. 23, 2004 (JP) 2004-084992

(51) Int. Cl. A61K 35/28; A61P 1/16; A61P 35/00; C12N 15/63; C12N 5/10

(52) U.S. Cl. 424/93.21; 435/325; 435/455

(57) ABSTRACT

The present invention provides transformed bone marrow-related cells that are associated with tissue maintenance and/or repair. Further, the invention provides methods for diagnosing and treating diseased tissues using the transformed bone marrow-related cells. The transformed bone marrow-related cells of the present invention are transformed bone marrow-related cells that are introduced with gene-carrying vectors and that are associated with tissue maintenance and/or repair. Moreover, the methods for preparing the transformed bone marrow-related cells of the present invention comprise the step of using gene-carrying vectors to introduce genes to bone marrow-related cells taken from mammals.

### TECHNICAL FIELD

The present invention relates to transformed bone marrow-related cells, which are introduced with gene-carrying vectors and associated with tissue maintenance and/or repair, and uses of these cells.

### BACKGROUND ART

The predominant therapeutic goals of conventional medicine were early detection of diseased sites such as damaged organs and tissues, determining the causes of diseases, and removing damaged sites at early stages. Such therapies rely largely on the naturally healing (or recovery) abilities of the organs or tissues that remain after the removal. However, even when providing such therapies, if the amount removed exceeds a certain level, it is difficult for the organs or tissues to recover their original functions. Patients in such situations require organ or tissue regeneration therapy, or ex vivo supplementation by transplantation of organ or tissue.

Recently, fundamental study of regenerative medicine has progressed, and there is much hope of using stem cells to regenerate central nervous function or treat severe diseases such as muscular dystrophy and Parkinson's disease. Stem cells, which are the foundation of regenerative medicine, are hierarchical in their differentiation process, and less differentiated stem cells have a greater ability to self-replicate. It has been reported that bone marrow stem cells, which are comprised in bone marrow cells, are less differentiated cells, and have pluripotency and have the potential to differentiate into cells beyond the germ layer (Jiang et al., 2002). Bone marrow stem cells are already clinically applied in tissue engineering fields such as regeneration of bone, cartilage, slin, and the like using bone marrow cells (Kuroyanagi, 2003). In regenerative medicine, studies using both self and non-self cells have progressed, and strategies have been designed for performing cell therapy using vascular endothelial cells, cardiac myocytes, neuronal cells, or liver cells, which are induced to differentiate from autologous bone marrow cells as a donor source (Takahashi, 2002).

Organs and tissues are composed not only of parenchymal cells, but are also constructed by multiple components such as extracellular matrices which provide anchorage for cell adhesion, and non-parenchymal (or mesenchymal) cells. Therefore, treatment methods that aim to simply remove or cure disease-causing target cells make it difficult to restore organs and tissues to their original size and function.

Recently, cells derived from transplanted bone marrow cells were found in multiple organs and tissues of patients who had received bone marrow transplants, suggesting that bone marrow cells may play some role in tissue repair (Krause et al, 2001). Further, it has been reported that therapeutic efficacy may be enhanced by returning to the body, mesenchymal stem cells (MSCs) transfected ex vivo (Ohlsson et al., 2003). Thus, bone marrow stem cells and MSCs are likely to play very important roles in the regeneration and recovery of organs and tissues.
(Non-Patent Document 1) Jiang, Y. et al., Nature, 418,41-49(2002)
(Non-Patent Document 2) Yoshimitsu Kuroyanagi, Journal of The Japanese Society for Regenerative Medicine “Regenerative Medicine” Vol. 2, No. 3, 39-45(2003)
(Non-Patent Document 3) Jun Takahashi, Journal of The Japanese Society for Regenerative Medicine “Regenerative Medicine” Vol. 2, No. 2, 67-74(2002)
(Non-Patent Document 4) Krause, D. S. et al., Cell, 105, 369-377(2001)
(Non-Patent Document 5) Ohlsson, L. B. et al., Exp. Mol. Pathol., 75,248-255(2003)

### DISCLOSURE OF THE INVENTION

An objective of the present invention is to provide transformed bone marrow-related cells, which have been introduced with gene-carrying vectors and are associated with tissue maintenance and/or repair. Another objective of the present invention is to provide methods for preparing transformed bone marrow-related cells, comprising the step of using gene-carrying vectors to introduce genes into bone marrow-related cells taken from mammals.

The present invention enables diagnosis and therapy of diseases associated with the maintenance and/or repair of a living tissue by preparing transformed bone marrow-related cells introduced with gene-carrying vectors, and using the cells.

As described above, bone marrow stem cells and mesenchymal cells are considered to play crucial roles in regenerative medicine. Traditionally, therapies aimed at killing specific cancerous cells in the body, such as administering mesenchymal cells introduced with a particular gene into the body had peen performed. However, as opposed to methods for directly killing target cells, methods for treating diseases by maximizing the natural healing ability of organs and tissues are unknown. Therefore, the present inventors aimed to prepare transformed bone marrow-related cells introduced with specific genes, with the aim of diagnosing and treating diseases, targeting tissue maintenance and/or repair. Specifically, they introduced bone marrow-related cells with a vector carrying a gene related to tissue maintenance or repair, and administered the transformed bone marrow-related cells into disease model laboratory animals, thereby successfully restoring the functions of the diseased tissues and accomplishing the present invention. Thus, by providing transformed bone marrow-related cells introduced with gene-carrying vectors, the present invention assists in meeting the diverse needs of such cells in the field of regenerative medicine.

Thus, the present invention provides transformed bone marrow-related cells introduced with a gene-carrying vector, wherein the above transformed bone marrow-related cells are associated with tissue maintenance and/or repair. More specifically, the present invention relates to the following inventions:
[1] a transformed bone marrow-related cell introduced with a vector carrying a gene, wherein the cell is associated with the maintenance and/or repair of a tissue;
[2] the transformed bone marrow-related cell of [1], wherein the gene is a marker gene, or has a function of directly participating in the maintenance and/or repair of a tissue, or of assisting a function of the transformed bone marrow-related cell in maintaining and/or repairing a tissue;
[3] the transformed bone marrow-related cell of [2], wherein the gene with the function of directly participating in the maintenance and/or repair of a tissue, or of assisting a function of the transformed bone marrow-related cell in maintaining and/or repairing a tissue, encodes a protein or a peptide having an activity of controlling the differentiation or proliferation of a cell or of controlling a cellular function, wherein the protein or the peptide is selected from the group consisting of HGF, FGF, VEGF, PDGF interleukin, GCSF, MCSF, SCF, IFN, Crx, and Otx2;
[4] the transformed bone marrow-related cell of any one of [1] to [3], wherein the vector is an adenoviral vector or a Sendai virus vector;
[5] the transformed bone marrow-related cell of [4], wherein the adenoviral vector carries an HGF gene;
[6] the transformed bone marrow-related cell of [4], wherein the Sendai virus vector carries an FGF2 gene;
[7] the transformed bone marrow-related cell of [4], wherein the Sendai virus vector carries an IFN gene;
[8] the transformed bone marrow-related cell of any one of [1] to [7], wherein the bone marrow-related cell is a bone marrow cell or a bone marrow-derived cell;
[9] the transformed bone marrow-related cell of any one of [1] to [8], wherein the tissue is a diseased tissue;
[10] the transformed bone marrow-related cell of [9], wherein the disease is a liver disease;
[11] the transformed bone marrow-related cell of [10], which reduces a level of a serum liver enzyme;
[12] the transformed bone marrow-related cell of [9], wherein the disease is a cancer;
[13] the transformed bone marrow-related cell of [12], wherein the cancer is a hepatic cancer;
[14] the transformed bone marrow-related cell of any one of [1] to [13], for injection into a peripheral blood vessel;
[15] a method for preparing a transformed bone marrow-related cell, comprising the step of using a vector carrying a gene to introduce the gene to a bone marrow-related cell taken from a mammal;
[16] use of a recombinant vector carrying a gene for preparing a transformed bone marrow-related cell;
[17] a pharmaceutical agent for the maintenance and/or repair of a tissue, comprising the transformed bone marrow-related cell of any one of [1] to [14];
[18] an agent for treating a liver disease, comprising the transformed bone marrow-related cell of [10];
[19] the agent for treating a liver disease of [18], wherein the liver disease is a hepatopathy, hepatic insufficiency, cirrhosis, or hepatitis;
[20] the agent for treating a liver disease of [18], wherein the liver disease is a hepatic cancer;
[21] the agent for treating a liver disease of [18] or [19], wherein the gene is an HUG or an FGF2;
[22] the agent for treating a liver disease of [18] or [20], wherein the gene is an IFN;
[23] the agent for treating a liver disease of any one of [18] to [22], wherein the vector is an adenoviral vector or a minus-strand RNA viral vector;
[24] the agent for treating a liver disease of [23], wherein the vector is a minus-strand RNA viral vector deficient in the F gene;
[25] a method for manufacturing an agent for treating a liver disease, comprising the step of preparing a composition comprising the transformed bone marrow-related cell of [10] and a pharmaceutically acceptable medium;
[26] the method of [25], wherein the liver disease is a hepatopathy, hepatic insufficiency, cirrhosis, or hepatitis;
[27] the method of [25], wherein the liver disease is a hepatic cancer;
[28] the method of [25] or [26], wherein the gene is an HGF or FGF2;
[29] the method of [25] or [27], wherein the gene is an IFN;
[30] the method of any one of [25] to [29], wherein the vector is an adenoviral vector or a minus-strand RNA viral vector; and
[31] the method of [30], wherein the vector is a minus-strand RNA viral vector deficient in the F gene.

The genes introduced into the transformed bone marrow-related cells of the present invention comprise marker genes, or comprise genes-with a function of directly participating in tissue maintenance and/or repair, or of assisting the function of the transformed bone marrow-related cells in tissue maintenance and/or repair. The transformed bone marrow-related cells of the present invention may be used as medicines for promoting tissue maintenance and/or repair.

The vectors carrying the genes to be introduced into the transformed bone marrow-related cells of the present invention are generally not particularly limited, as long as they allow expression of genes in cells derived from mammals such as humans and mice. The vectors are preferably viral vectors, in particular, recombinant adenoviral vectors or minus strand RNA viral vectors, such as Sendai virus vectors. Preferable embodiments of the above gene-carrying vectors are adenoviral vectors or Sendai virus vectors carrying HGF, FGF-2, or IFNβ genes. Particularly preferable embodiments of the above gene-carrying vectors are adenoviral vectors carrying the HGF gene (adexHGF), F-gene deleted Sendai virus vectors carrying the FGF-2 gene (FGF2-SeV/ΔF), or F-gene deleted Sendai virus vectors carrying the IFNβ gene (IFNβ-SeV/ΔF), and more preferably adexHGF, FGF2-SeV/ΔF, and IFNβ-SeV/ΔF. In an embodiment of the present invention, hFGF2-SeV/ΔF and IFNβ-SeV/ΔF may be used as the recombinant Sendai virus vectors for humans.

The bone marrow-related cells used herein include bone marrow cells and bone marrow-derived cells.

The tissues which are maintained and/or repaired by the transformed bone marrow-related cells of the present invention are typically diseased tissues. Further, when the objective is to maintain original tissue functions, the tissues are not limited to diseased tissues. Diseased tissues are those suffering from an inflammatory disease, hepatic disease, immune disease, cancer, genetic disease, or the like; preferably an inflammatory disease, hepatic disease, or cancer; and more preferably an inflammatory disease or hepatic disease. Thus, the transformed bone marrow-related cells of the present invention can be used as drugs for treating diseases such as inflammatory diseases, hepatic diseases, immune diseases, cancers, and genetic diseases. More preferably, the transformed bone marrow-related cells of the present invention can be used as drugs for treating hepatic diseases. The cells may be prepared as medicinal compositions by appropriate combination with pharmaceutically acceptable media. The present invention filter relates to methods for manufacturing the above drugs for disease treatments, comprising the step of introducing gene-carrying vectors into bone marrow-related cells, and uses of the above vectors or transformed bone marrow-related cells in manufacturing the above drugs for disease treatments. Favorable genes are HGF, FGF2, or IFNβ.

In an embodiment of the present invention, the level of serum liver enzymes can be reduced by using the transformed bone marrow-related cells of the present invention. Thus, the transformed bone marrow-related cells of the present invention can be used as agents for reducing the level of serum liver enzymes. The present invention further relates to methods for producing agents for reducing the level of serum liver enzymes, comprising the step of introducing gene-carrying vectors into bone marrow-related cells, and uses of the above vectors or transformed bone marrow-related cells in the production of agents for reducing the level of serum liver enzymes. Preferable genes are HGF or FGF2.

In another embodiment of the present invention, the growth of cancers such as hepatic cancers can be suppressed by using the transformed bone marrow-related cells of the present invention. Thus, the transformed bone marrow-related cells of the present invention can be used as therapeutic agents for cancers such as hepatic cancer. The present invention further relates to methods for producing therapeutic agents for cancers (or agents for suppressing cancer cell growth) comprising the step of introducing gene-carrying vectors into bone marrow-related cells; and to uses of the above vector or transformed bone marrow-related cells in producing therapeutic agents for cancers (or agents for suppressing cancer cell growth). Preferable genes are IFNs, and IFNβ in particular.

The transformed bone marrow-related cells of the present invention may be administered into peripheral blood vessels or administered subcutaneously, intramuscularly, intraperitoneally, intratracheally, intraventricularly, intraspinally, or intrathoracically, but these are not limiting. The transformed bone marrow-related cells are preferably administered into peripheral blood vessels or subcutaneously administered, and are more preferably administered into peripheral blood vessels.

The present invention provides methods for preparing the transformed bone marrow-related cells of the present invention. The methods of the present invention typically comprise the steps of collecting bone marrow-related cells from mammals, and introducing genes into the cells using vectors carrying, those genes.

The present invention provides methods of using recombinant gene-carrying vectors for preparing transformed bone marrow-related cells.

### BEST MODE FOR CARRYING OUT THE INVENTION

The present invention is explained below with reference to preferable embodiments.

### (1) Bone Marrow-Related Cells

Bone marrow-related cells used for the transformed bone marrow-related cells of the present invention include bone marrow cells and bone marrow-derived cells, and other hematopoietic stem cells and cell components in the blood. Bone marrow cells or bone marrow-derived cells are preferable. Bone marrow cells compose bone marrow, and are a group of precursor cells involved in hematopoiesis. Thus, bone marrow cells comprise many hematopoietic stem cells, for example, CD34 positive cells. Erythrocytes, which are in the final stage of differentiation, are generated from hematopoietic stem cells through differentiation into protoerythrocytes, prorubricytes, polychromatophilic erythroblasts, and normoblasts. Leukocytes are classified into neutrophilic leukocytes, eosinophilic leukocytes, basophilic leukocytes, lymphocytes, monocytes and such, according to granule staining properties. In particular, lymphocytes comprise cells that mature in the bone marrow, and cells that mature in the spleen. Cells that mature in the spleen are considered T cells, and cells that mature in the bone marrow are considered B cells.

Herein, “bone marrow cells” refer to precursor cell group involved in hematopoiesis among the cells composing the bone marrow, as described above, and is not limited to refer to particular cells, while large numbers of the above hematopoietic stem cells normally exist in bone marrow, they are also known to appear in the peripheral blood during the recovery of hematopoiesis in bone marrow after chemotherapy, or after the use of C-CSF (granulocyte colony stimulating factor). Such hematopoietic stem cells are specifically called peripheral blood stem cells. In an embodiment of the present invention, bone marrow-related cells also include peripheral blood stem cells. Hematopoictic stem cells are also known to be present in umbilical cord blood, and these umbilical cord blood hematopoietic stem cells are also included in the bone marrow-related cells. Hematopoietic cells such as peripheral blood hematopoietic stem cells, umbilical cord blood hematopoietic stem cells, and bone marrow cells are suitable for the use in the present invention.

Herein, “bone marrow-derived cells” comprise B cells that are precursor cells of antibody-producing cells originating from bone marrow. B cells are cells that differentiate from stem cells in the liver during prenatal period, and in the bone marrow after birth. During differentiation, the immunoglobulin (Ig) gene locus is activated and recombinase gene products are expressed, inducing the immunoglobulin heavy (IgH) chain to rearrange, and expressing the μ chain. Cells at this stage are called pre-B cells. In pre-B cells, μ chain expression is considered to play an important role in the subsequently induced B cell maturation. Further, B cells in which L chain rearrangement is induced will differentiate into antibody-producing cells upon antigen stimulation. Besides B cells, bone marrow-derived cells also include a variety of cell components in the blood, such as platelets, erythrocytes, granulocytes, T cells, and the like.

To obtain bone marrow-related cells, pluripotent stem cells must be isolated from other types of cells in bone marrow or other hematopoietic sources. Bone marrow cells may be obtained from bone marrow sources, for example, the iliac crest, tibia, thighbone, vertebral column, or other bone cavities. Hematopoietic stem cells may be also obtained from other sources such as yolk sacs in embryos, fetal liver, fetal and adult spleens, and blood such as adult peripheral blood and umbilical cord blood. To isolate bone marrow from fetal bones or other bone sources, a balanced salt solution appropriate for washing bone marrow out from bones may be used. In general, a balanced salt solution containing an acceptable buffer at low concentrations such as around 5-25 mM, supplemented with fetal bovine serum or other natural factors, may be used. Preferable buffers are Hepes, phosphate buffer, or lactate buffer. In another method, bone marrow may aspired from bones, according to standard methods. For example, bone marrow cells may be collected from thighbones or tibiae of mice according to the method described by Terai S. et al. (J. Biochem. 134: 551-558 (2003).

Bone marrow-derived cells may be isolated from bone marrow obtained by an above method, by using antigens presented on the membrane surface of the bone marrow-derived cells as a marker. An example of such equipment used to isolate bone marrow-derived cells is FACS (Becton, Dickinson and Company). Alternatively, bone marrow-derived cells may also be isolated by adsorbing molecules that bind to antigens presented on the cell surface onto magnetic beads or such.

Specifically, the bone marrow-related cells used in the present invention include cells from vertebrates, preferably cells derived from mammals. For example, the bone marrow-related cells include cells derived from humans, mice, Xenopus laevis, rats, hamsters, or monkeys, or established cultured cell lines from these cells.

Hematopoietic stem cells (HSCs) in bone marrow-related cells can be identified by using colony assays to count the number of precursor cells for granulocytes and monocytes, or by using flow cytometry to determine CD34 (antigen) positive cells, thought to be characteristic of HSCs. Specific examples of HSCs are lineage negative (lin⁻) cells with the phenotype of CD34⁺ or CD38⁻ (Bhatia M. et al. Proc. Natl. Acad. Sci. U.S.A. 94: 5320-5325 (1997). The lin⁻ (lineage negative) phenotype can be appropriately identified and selected using commercial kits and the like; for example, GPA, CD3, CD2, CD56, CD24, CD19, CD14, CD16, and CD99b are all negative.

HSCs from humans may be isolated according to the methods described in the following references: (Leary, A G., Blood 69:953, 1987; Sutherland H J, Blood 74:1563, 1989; Andrews R G; J Exp Med 169:1721, 1989; Terstappen L W M M, Blood 77:1218, 1991;3 Lansdorp P M, J Exp Med 175:1501, 1992; Briddell R A, Blood 79:3159, 1992; Gunji Y, Blood 80:429, 1992; Craig W, J Exp Med 177:1331, 1993; Gunji Y, Blood 82:3283, 1993; Traycoff C N, Exp Hematol 22:215, 1994; Huang S, Blood 83:1515,1994; DiGinsto D, Blood 84:421, 1994; Murray L, Blood 85:368, 1995; Hao Q L, Blood 86:3745, 1995; Layer J H, Exp Hematol 23:1515, 1995; Berardi A C, Science 267:104, 1995; Kawashima I, Blood 87:4136, 1996; Leemhuis T, Exp Hematol 24:1215, 1996; Civin C I, Blood 88:4102, 1996; Larochelle A, Nature Med 2: 1329, 1996; Tajima S, J Exp Med 184:1357, 1996; Sakabe H, Stem Cells 15:73, 1997; Sakabe H, Leukemia 12:728, 1998; Harada M et al., eds., “Atarashii zouketu kansaibou ishoku (Novel hematopoietic stem cell transplantation)”, Nankodo, 1998, pp 9-23).

As a more specific example of methods for obtaining bone marrow cells, for example, normal hematopoiesis is confirmed in bone marrow, then bone marrow cells are collected from iliac bones and breastbones under general anesthesia. The target cell number is around 3×10⁸ to 5×10⁸ nucleated bone marrow cells/kg. Cells are collected a few ml at a time, over multiple times. In case of contamination by bone tissue or such, cells may be passed through mesh, and then sealed in a bag. Cells can be transplanted by transfusion using coarse filters. For allogenic transplantation, an HLA (human lymphocyte antigen)-matched donor is preferably selected. However, the establishment of methods for preventing GVID (graft-versus-host disease), the development of novel immune suppressing agents, techniques for purification of CD34 positive cells and such have made it possible to perform transplantation from an HLA-mismatched donor. A donor preferably matches a recipient in four or more of the six antigens in the three classes HLA- A, HLA- B, and HLA- DR, more preferably five or more, and most preferably all of the six antigens.

Peripheral blood stem cells (PBSCs) may be collected from peripheral blood by subcutaneously injecting G-CSF every day, and collecting mobilized peripheral blood CD34⁺ cells when their cell number peaks. Normally, mobilization of CD34 positive cells lasts for several days, and thus PBSCs can be collected every day. For mobilization and collection of PBSCs, see the following references: Harada M. et al. J. Hematother. 5: 63-71 (1996); Waller C. F. et al. Bone Marrow Transplant. 18: 279-283 (1996); Anderlini P. et al. Blood 90: 903-908 (1997); “Atarashii zouketu kansaibou ishoku (New methods of hematopoietic stem cell transplantation)” Mine Harada et al. edit. Nankodo pub. p67-72 (1998); “Zouketu saibou ishoku manual (Manual for hematopoietic cell transplantation)” revised third edition, Nagoya BMT Group edit. p237-240 (2004). The G-CSF used for mobilization may be wild type proteins, or N-terminal modified derivatives (nartograstin, etc.), or proteins modified by sugar chain addition (lenograstin, etc.). G-CSF may be also used in combination with other hematopoietic factors. For example, GM-CSF, IL-3, or SCF may be used in combination with G-CSF (Huhn R. D. et al. Exp. Hematol. 24: 839-847 (1996); Begley C. G. et al. Blood 90: 3378-3389 (1997); Lane T. A. et al. Blood 85: 275-282 (1995)). During G-CSF administration, aspirin may be administered to reduce systemic symptoms, such as lower-back pain, ostealgia, and fever, and to prevent excessive platelet aggregation.

Platelet contaminants, if transfused as is, sometimes cause embolus and such, and thus, they are preferably removed after collection. For example, collected cells may be packed in double bags and centrifuged at low speed (200 g, 15 minutes), or dispensed into centrifugation tubes and centrifuged at 1600 rpm for 10 minutes to remove platelets and exchange the medium with RPMI1640 medium. For cryopreservation, collected cells are suspended in RPMI1640 medium containing 10% autologous serum and 10% DMSO and then frozen by a programmed freezer, and stored in liquid nitrogen. The concentration of cells can be from 2×10⁷ to 6×10⁷ cells/ml. For cell storage over a relatively short period, a cell suspension (1×10⁸ cells/ml or a lower concentration) can be mixed with an equal volume of ice-cold storage solution to a final concentration of 6% Hydrodyethyl Starch (HES), 5% DMSO, and 4% albumin, and cryopreserved at −80° C. in a deep freezer (Knudsen L. M. et al. J. Hematother. 5: 399-406 (1996)).

To prepare umbilical cord blood stem cells, erythrocyte precipitating agent (HES) is added to umbilical cord blood, the upper layer is recovered in a fresh bag, and this is centrifuged to separate and concentrate cells, Collected cells can be preserved by adding a cryoprotective solution and freezing in a programmed freezer.

Cell suspensions obtained using a continuous-flow apheresis system have very little granulocyte and erythrocyte contamination, and thus the procedure for separating monocytes may be omitted. If many granulocytes and erythrocytes are comprised, monocytes can be separated by Ficoll specific gravity centrifugation. In the case of bone marrow suspensions, monocytes may be separated by Ficoll treatment or by an apheresis system, to remove granulocytes and erythrocytes and concentrate the monocytes. CD34 positive cells may be purified from the obtained cell fractions by using, for example, an Isolex system (Nexell) or CliniMACS (AmCell).

Isolated bone marrow-related cells may be cultured by methyl cellulose methods by adding SCF, IL-3, GM-CSF, G-CSF, and Epo (Sonoda Y et al. Blood 84: 4099-4106 (1994); Kimura T. et al. Blood 90: 4767-4778 (1997)). Cells are added at around 1×10² to 1×10⁴ cells/ml, and cultured at 37° C., 5% CO₂, and 5% O₂, for example. However, culture conditions may be appropriately adjusted.

The number of CD34 positive cells used for autologous transplantation may be about 2×10⁶ cells/kg, for example (Schots R. et al. Bone Marrow Transplant. 17: 509-515 (1996); Zimmerman T. M. et al. Bone Marrow Transplant. 15: 439-444 (1995)). The number of CD34 positive cells can be measured using standard two-color flow cytometry. Specifically, erythrocytes are removed by hemolytic treatment, from bone marrow cells or peripheral blood cells that underwent apheresis, then developed using forward scatter and anti-CD45 antibody, and then erythrocytes and platelets can be removed by gating. The gated fraction is then developed using the side scatter and anti-CD34 antibody to remove non-specifically reacting fractions containing neutrophils and such, and the number of cells in the remaining fraction as compared to the total cell number is calculated. This value and the number of blood cells predetermined using a hemocytometer is used to calculate the total number of CD34 positive cells.

The number of viable stem cells comprised in cryopreserved cells can be determined by the method of counting CFU-GMs using colony formation methods. The standard for CFU-GMs required for transplantation is generally 1×10⁵ to 2×10⁵ cells/kg.

### (2) The Transformed Bone Marrow-Related Cells of the Present Invention

The transformed bone marrow-related cells of the present invention are transformed bone marrow-related cells introduced with a gene-carrying vector.

The genes used for transformation of bone marrow-related cells comprise marker genes, or comprise genes that directly participate in tissue maintenance and/or repair, or that function to support the function of transformed bone marrow-related cells in maintaining and/or repairing tissues.

Genes that directly participate in tissue maintenance and/or repair, or that function to support the function of transformed bone marrow-related cells in maintaining and/or repairing tissues may be any genes that can be expressed in transformed bone marrow-related cells, where the products of these genes are directly useful in tissue maintenance and/or repair. Alternatively, these gene products can have the function of directly or indirectly supporting the intrinsic function of bone marrow-related cells in maintaining and/or repairing tissues. Embodiments of the genes that can be used in the present invention include genes encoding proteins or peptides that have the activities of regulating cell differentiation, cell proliferation, and a variety of cellular functions, selected from the group consisting of HGF, FGF, VEGF, PDGF, interleukin, G-CSF, M-CSF, SCF, IFN, Crx, and Otx2. Information on the nucleotide sequences of these genes can be obtained from gene databases NCBI, for example). Thus, those skilled in the art can use the obtained genetic information to integrate these genes into expression vectors or the like.

For example, hepatocyte growth factors (HGF) are reported by Miyazawa et al., Biochem. Biophys. Res. Comnm. 163,967-973, 1989; Nakamura et al., Nature, 342, 440-443, 1989; Seki et al., Biochem. Biophys. Res. Comm. 172, 321-327, 1990; Tashiro et al., Proc. Natl. Acad. Sci, USA, 87, 3200-3204, 1990; Okajima et al., Eur. J. Biochem. 193, 375-381, 1990; Nakamura et at. J. Clin. Invest. 106, 1511-1159, 2000. Many HGF variants are known in addition to natural polypeptides. For example, genes encoding the HGFs described in Japanese Patent Application Kokai Publication No. (JP-A) H5-111383; U.S. Pat. Nos. 4,683,195, 4,816,567, 4,745,055, and 4,444,878; European Patent Nos. 256654, 120694, 125023, 255694, and 266663; WO 88/03559, WO 88/03565, and WO 94/06456 and their derivatives may be used. Furthermore, HGFs wherein the human HGF amino acid has been substituted at positions 534, 673, and/or 692 may be used (JP-A 2004-000236). The nucleotide sequence of the HGF gene can be found in Accession No.: NM_{—}000601 (nucleotides 166 to 2349), and at 166 to 2334 of AccessionNo.: NM_{—}001010932. The amino acid sequence of HGF can be found in Accession No.: NP_{—}000592 and NP_{—}001010932. Furthermore, fusion peptides to which desired polypeptides such as immunoglobulin constant regions or fibronectin fragments have been added may be used as HGFs (JP-A 2004-269423, WO 91/08298).

Fibroblast growth factor 2 (FGF2), also called basic fibroblast growth factor (bFGF), is known as a factor comprising the activity of promoting not only the growth of fibroblasts, but also the growth of a variety of cells such as vascular endothelial cells, cartilage cells (chondrocytes), osteoblasts, and epidermal cells (Abraham et al. EMBO J. 5: 2523-2528 (1986); Prats et al. Proc. Natl. Acad. Sci. U.S.A. 86: 1836-1840 (1989)). For example, the nucleotide sequence of FGF2 gene and its amino acid sequence can be found in Accession number: NM_{—}002006 (nucleotides 69-932), and in Accession number: NP_{—}001997, respectively. FGF2 includes not only natural proteins, but also products genetically engineered using recombinant DNA technology, and modified forms of those. For example, those described in WO 87/01728, WO 89/04832, WO 86/07595, WO 87/03885; EP Patent applications No. 237966, No. 281822, No. 326907, No. 394951, No. 493737 and such may be used.

For interferons (IFN), for example, see Gren et alt (1984) J. Interferon Res. 4(4):609-617, and Weismann et al. (1982) Princess Takamatsu Symp. 12:1-22, regarding IFN-α, and Derynck, R. et at., Nature 285, 542-547 (1980); Higashi, Y. et al., J. Biol. Chem. 258, 9522-9529 (1983); Kuga, T. et al., Nucleic Acids Res. 17, 3291 (1989) regarding IFN-β. For example, the nucleotide sequence of IFN-α1 gene can be found in Accession No.: NM_{—}024013 (nucleotides 68 to 634), and the amino acid sequence of IFN-α1 is described in Accession No.: NP_{—}076918. The nucleotide sequence of IFN-β gene can be found in Accession No.: NM_{—}002176 (nucleotides 76 to 636), and the amino acid sequence of IFN-β is described in Accession No.: NP_{—}002167. The nucleotide sequence of IFN-γ gene can be found in Accession No.: NM_{—}000619 (nucleotides 127 to 624), and the amino acid sequence of IFN-γ is described in Accession No.: NP_{—}000610. There are polymorphs and variants of the above cytokines. As long as the polymorphs and variants have activities equivalent to a wild type cytokine, they may be appropriately used. The IFNs of the present invention comprise IFN-α, IFN-β, IFN-γ, and such, but are preferably type I IFNs (IFN-α and -β).

The biological activities of various cytokines and their derivatives may be assayed using known methods. -For example, the activity of HOF may be determined by detecting the in vitro or inl vivo promotion of hepatocyte proliferation. Specifically, by adding natural or artificial HGFs to primary cultured hepatocytes and detecting the promotion of cellular DNA synthesis, their action to promote cellular division in hepatocytes can be identified. DNA synthesis in hepatocytes can be assayed by the uptake of [³H]-thymidine (Nakamura, Biochem. Biophys. Res. Com. 122: 1450-1459 (1984); Nakamura, J. Biochem. 94: 1029-1035 (1983)). In case of FGF2, the activity of promoting proliferation of fibroblasts or vascular endothelial cells can be determined in the same way as described above. In case of IFNs, anti-viral activity can be measured by assaying the activity of inhibiting the cytotoxicity of vesicular stomatitis virus. Specifically, WISH cells (CCL-25; A.T.C.C. (American Type Culture Collection), Manassas, Va., U.S.A.) are inoculated with vesicular stomatitis-Indiana-virus (VR-1238 AF; ATCC), and virus-induced cell death may be detected to determine IFN-mediated protection (according to measurement conditions described in Knezic Z. et al. Antiviral Res. 25: 215-221 (1993)).

Herein, “the activity of regulating cell differentiation and proliferation” means the activity of quantitatively controlling the cells to increases the number of cells with a desired function.

Herein, “the activity of regulating cellular functions” means the activity of qualitatively controlling the cells to enhances a desired function in pre-existing cells.

Herein, “a factor related to immune suppressione” refers to a protein or peptide with the activity of weakening or avoiding an immune reaction or of actively inducing immune tolerance.

Herein, “a marker” refers to a gene encoding a protein suited to immunohistochemical staining, or direct or indirect immunofluorescent staining of tissues or cells. Such markers are preferably GFP, Liv2, HNF4, A6, albumin, luciferase, β-galactosidase, or SEAP, more preferably, GFP, luciferase, or β-galactosidase, and most preferably GFP. GFP is an abbreviation of green fluorescent protein.

These marker genes can be expressed in transformed bone marrow-related cells, and can be detected using known detection methods, according to the kind of marker gene. For example, GFP is detectable by fluorescence in the range of 490 nm to 520 nm. Thus, by using direct or indirect immunofluorescent stalning, its localization in tissues of the above bone marrow cells can be observed using fluorescence microscope.

Furthermore, GFP mutants which emit stronger fluorescence such as enhanced green fluorescent protein (EGFP), yellow fluorescent protein (YFP), blue fluorescent protein (BFP), and red fluorescent protein (REP) (available from Clontech, for example) may also be used as markers.

Vectors that can integrate genes into bone marrow-related cells are, briefly, expression vectors obtainable in this technical field. The types of expression vectors are not limited to any specific type, as long as they have the function of producing desired proteins by expressing desired genes in bone marrow-related cells. For example, they include viral vectors, plasmid vectors, phage vectors, etc. In particular, minus strand RNA viral vectors such as adenoviral vectors and Sendai virus vectors, and retroviral vectors such as adeno-associated viral (AAV) vectors and lentiviral vectors are preferably used viral vectors. Minus strand RNA viral vectors such as adenoviral vectors and Sendai virus vectors are most preferable.

Genes can be introduced and expressed using viral vectors according to the method described in “Shin idenshikougaku handbook (New edition of the handbook on genetic engineering)” revised edition, Muramatsu and Yamamoto edit. Experimental Medicine, supplementary volume, Yodosha, p202-215 (1999). Viral vectors are known to have high expression efficiency in cultured animal cells. As described in the following Example 1, gene expression of both adenoviral vectors and Sendai virus vectors could be observed in 80% or more bone marrow cells. By using these viral vectors, genes can be expressed without killing the gene-introduced cells, thus enabling analysis of the functions of those genes. While it is known that methods for preparing recombinant viral vectors are extremely inefficient, preparation efficiency may be improved by employing the COS-TPC method (Miyake S. et al. Proc. Natl. Acad. Sci. U.S.A. 93: 1320-1324 (1996)), for example,

To give a farther example, adenoviral vectors may be prepared according to methods described by Saito et al. and others (Miyake et al. Proc. Natl. Acad. Sci. U.S.A. 93: 1320-1324 (1996); Kanegae et al. Acta Paediatr. Jpn. 38: 182-188 (1996); Kanegae et al. “Biomanual Series 4, Idenshi dounyuu to hatsugen kaisekihou (Methods of gene introduction, expression, and analysis)” Yodosha, p43-58 (1994); Kanegae et al. Cell Technology 13 (8): 757-763 (1994)). Vectors such as retroviral vectors may be prepared by the method of Wakimoto et al. (Protein, Nucleic acid and Enzyme 40: 2508-2513 (1995)), and AAV vectors may be prepared by the method of Tamayose et al. (Protein, Nucleic acid and Enzyme 40: 2532-2538 (1995)). Methods for producing other viral vectors capable of introducing genes into mammals are described in detail in the following: methods for preparing recombinant vaccinia viruses, Japanese Patent Kohyo Publication No. (JP-A) H6-502069 (unexamined Japanese national phase publication corresponding to a non-Japanese international publication), and Japanese Patent Application Kokoku Publication No. (JP-B) H6-95937 (examined, approved Japanese patent application published for opposition) and JP-B H6-71429; recombinant papilloma viruses, JP-B H6-34727 and JP-A H6-505626; recombinant adeno-associated viruses, JP-A H5-308975; recombinant adenoviruses, JP-A H6-508039; and minus strand RNA viruses, WO 97/16539, WO 97/16538, WO 00/70055, and WO 00/70070.

The minus strand RNA viruses used herein are particularly preferably single-stranded minus-strand RNA viruses (also referred to as non-segmented minus-strand RNA viruses), which have a single-stranded negative strand [i. e., a minus strand] RNA as the genome. Such viruses include viruses belonging to Paramyxoviridae (including the genera Paramyxovirus, Morbillivirus, and Rubulavirus), Rhabdoviridae (including the genera Vesiculovirus. Lyssavirus, and Ephemerovirus). Filoviridae, Orthomyxoviridae, (including Influenza viruses A, B, and C, and Thogoto-like viruses), Bunyaviridae (including the genera Bunyavirus, Hantavirus, Nairovirus, and Phlebovirus), Arenaviridae, and the like.

The minus strand RNA viruses used in the present invention are more preferably those belonging to Paramyxovirinae (including Respirovirus, Rubulavirus, and Morbillivirus) or derivatives thereof and more preferably those belonging to the genus Respirovoris (also called Paramyxovirus) or derivatives thereof. The derivatives include viruses that are genetically or chemically modified so as not to impair their gene-transferring ability. Examples of viruses of the genus Respirovirus applicable to is invention are human parainfluenza virus-1 (HPIV-1), human parainfluenza virus-3 (HPIV-3), bovine parainfluenza virus-3 (BPIV-3), Sendai virus (also referred to as murine parainfluenza virus-1), and simian parainfluenza virus-10 (SPIV-10). Sendai virus is the most preferable Paramyxovirus in the present invention. These viruses may be derived from natural strains, wild type strains, mutant strains, laboratory-passaged strains, artificial constructed strains or the like.

In the present invention, minus strand RNA viral vectors are preferably those deficient in one or more of the genes of envelope-constituting proteins. Envelope-constituting proteins refer to viral proteins which are components of the viral envelope, including spike proteins, which are exposed on the envelope surface and function in cell adhesion or infection, and lining proteins, which function in envelope formation and the like. Typically, envelope-constituting protein genes include F (fusion), HN (hemagglutinin neuraminidase), and M (matrix) genes. Some viral species have H (hemagglutinin), M1, G genes, etc. Viruses in which one or more of these envelope-constituting protein genes are mutated and/or deleted have a reduced ability to form infectious viral particles in infected cells, and are therefore safer. Such viruses also have significantly reduced cytotoxicity. Genes to be made deficient in the viral genome may be, for example, the F gene, HN (or H) gene, or M gene, or any combination of those. Most preferably, the viral genome is deficient in the F gene. The phrase “deficient in the F gene” means that the genome is at least deficient in the F gene, and may also be deficient in other genes. More preferably, the genome is deficient in at least the F and HN (or H) genes. More preferably, the genome is deficient in the F, HN (or H), and M genes.

More specific methods for reconstitution of recombinant minus-strand RNA viral vectors can be found in the following references: (WO) 97/16539; WO 97/16538; WO 00/70055; WO 00/70070; WO 01/18223; WO 03/025570; Durbin, A. P. et al., Virology, 1997:235; 323-332; Whelan, S. P. et al., Proc. Natl. Acad. Sci. USA, 1995:92; 8388-8392; Schnell. M. J. et al., EMBO J., 1994:13; 4195-4203; Radecke, F. et al., EMBO J., 1995:14; 5773-5784; Lawson, N. D. et al., Proc. Natl. Acad. Sci. USA, 92:4477-4481; Garcin, D. et al, EMBO J., 1995:14; 6087-6094; Kato, A. et at., Genes Cells., 1996:1; 569-579; Baron, M. D. and Barrett, T., J. Virol. 1997:71; 1265-1271; Bridgen, A. and Elliott, R. M., Proc. Natl. Acad. Sci. USA., 1996:93; 15400-15404; Hasan, M. K. et al., J. Gen. Virol., 1997:78; 2813-2820, Kato, A. et al., EMBO J., 1997:16; 578-587, Yu, D. et al., Genes Cells, 1997:2; 457-466, Tokusumi, T. et al., Virus Res., 2002:86; 33-38, Li, H. -O. et al., J. Virol., 2000:74; 6564-6569; Hirata, T. et al., J. Virol. Methods, 2002:104; 125-133; Inoue, M. et al., J. Virol., 2003:77; 6419-6429; Inoue, M. et al., J. Gene Med. 2004; 6:1069-1081. Using these methods, minus-strand RNA vises including parainfluenza viruses, vesicular stomatitis viruses, rabies viruses, measles viruses, rinderpest viruses, Sendai virus, and the like can be reconstituted from DNAs.

Preferred embodiments of the recombinant virus vectors are adexHGF (an adenoviral vector carrying an HGF gene), FGF2-SeV/ΔF (a Sendai virus vector which lacks an F-gene fragment and carries an FGF2 gene), and IFNβ-SeV/ΔF (a Sendai virus vector which lacks an F-gene fragment and carries an IFNβ gene). More preferably, they are adexHGF, hFGF2-SeV/ΔF, and IFNβ-SeV/ΔF. Specifically, the transformed bone marrow-related cells of the present invention may be prepared by infection with adexHGF, hFGF2-SeV/ΔF, IFNβ-SeV/ΔF or the like, as described in Example 2 below.

In an embodiment of the present invention, not only viral vectors but also recombinant vectors normally used for animal cells may be used. Methods for integrating genes into vectors such as plasmids include, for example, the methods described in Sambrook J. et al. Molecular Cloning, A Laboratory Manual (3rd edition), Cold Spring Harbor Laboratory, Chapter 1.1 (2001). Simply put, commercial ligation kits (for example, TAKARA) may also be used. Recombinant vectors (for example, recombinant plasmids) obtained in this way may be introduced into host cells (for example, E. coli DH5α, DH10BAC, XL-1Blue or the like) using methods such as calcium chloride methods, electroporation, and chemical treatment with PEG or such, and then amplified and purified (Sambrook J. et al. as described above). Recombinant expression vectors can be constructed according to the standard methods; for example, the Gateway system (Invitrogen), which does not require restriction enzyme treatments or ligation, may be used. The types of expression vectors are not particularly limited; however, for example, pFLAG-CMV-1, pcDNA3.1, pGreenLantem, and such may be preferably used as expression vectors for mammalian cells. The transformed bone marrow-related cells of the present invention may be obtained by introducing recombinant expression vectors into bone marrow-related cells.

In an embodiment of the present invention, tissues to be maintained and/or repaired include diseased tissues. Herein, a “disease” means a condition in which a part or all of an organ or tissue in the body is unable to sustain normal functions due to bacterial infection, viral infection, alcohol ingestion, drug administration, tumor development, abnormal gene expression, or the like, or patients with such conditions. Such diseases include, without limitation, inflammatory diseases, hepatic diseases, immune diseases, cancers, genetic diseases, and the like. In an embodiment of the present invention, inflammatory diseases include hepatic diseases. Such hepatic diseases are preferably hepatopathies, hepatic insufficiencies, hepatitises, cirrhoses, fatty liver, and hepatic cancers, and more preferably, hepatopathies, hepatic insufficiencies, and hepatitises. Thus, the present invention relates to methods for manufacturing drugs for the treatment of hepatic diseases by using vectors carrying genes with the fauction of assisting the function of tissue maintenance and/or repair by bone marrow-related cells, and uses of these vectors carrying genes with the function of assisting the function of tissue maintenance and/or repair by bone marrow-related cells in manufacturing drugs for the treatment of hepatic diseases, and the same uses of bone marrow-related cells introduced with these vectors. Administration of the transformed bone marrow-related cells of the present invention can reduce the measured levels of liver enzymes GOT, GPT, and LDH, and can improve liver functions. Thus, the present invention relates to methods for manufacturing agents for improving liver function by using vectors carrying genes with the function of assisting the function of tissue maintenance and/or repair by bone marrow-related cells, and uses of these vectors carrying genes with the function of assisting the function of tissue maintenance and/or repair by bone marrow-related cells in manufacturing the liver function improving agents, and the same uses of bone marrow-related cells introduced with these vectors. Examples of genes carried by such vectors are HGF, FGF2, and IFNβ, in particular.

Specifically, the present invention further comprises the following inventions:

(1) a method for manufacturing an agent for the treatment of a hepatic disease, comprising the step of introducing a vector encoding an HGF, FGF2, or IFN into a bone marrow-related cell;

(2) the method of (1), wherein the vector encodes an HGF or FGF2;

(3) the method of (1), wherein the vector encodes an IFN;

(4) the method of (2), wherein the hepatic disease is a hepatopathy, hepatic insufficiency, cirrhosis. or hepatitis;

(5) the method of (3), wherein the hepatic disease is a hepatic cancer;

(6) the method of any of (1) to (5), wherein the vector is an adenoviral vector or a minus-strand RNA viral vector;

(7) the method of (6), wherein the vector is an F-gene deficient minus-strand RNA viral vector;

(8) the method of (6) or (7), wherein the minus-strand RNA viral vector is a Sendai virus vector;

(9) an agent for treating a hepatic disease manufactured by the method of any of (1) to (8);

(10) an anti-cancer agent comprising a bone marrow-related cell introduced with a vector encoding an IFN, and a pharmaceutically acceptable medium.

(11) the anti-cancer agent of (10), wherein the cancer is a hepatic cancer.

(12) the anti-cancer agent of (10) or (11), wherein the vector is an adenoviral vector or a minus-strand RNA viral vector.

(13) the anti-cancer agent of (12), wherein the vector is an F-gene deficient niinus-strand RNA viral vector.

(14) the anti-cancer agent of (12) or (13), wherein the minus-strand RNA viral vector is a Sendai virus vector.

In an embodiment of the present invention, organs may also be the target for maintenance and/or repair using the transformed bone marrow-related cells of the invention.

### Examples of organs include livers, kidneys, and hearts.

In an embodiment of the present invention, tissues to be maintained and/or repaired include tissues after transplantation. Such transplanted tissues include skin tissues and tissues of various organs (for example, liver, kidney, and heart), but are not limited thereto. Furthermore, transplanted tissues may be tissues either from self or from non-self. Moreover, transplanted tissues are not limited to those taken from the body; they may also be cultured tissues grown by ex iivo culture of cells or a part of a tissue taken from the body, or artificially cultured tissues prepared by incorporating the cells or tissue into a biologically compatible material.

Herein, “tissue maintenance and/or repair” refers to maintenance of a function of a tissue or cells constituting the tissue, restoration to a certain level of an impaired function caused by a disease, or reconstruction (regeneration) of a tissue following removal of a diseased site. For example, in an embodiment of the present invention, the measured level of liver enzymes GOT, GPT, and LDH in hepatic diseases may be reduced. Herein, “to reduce the level of liver enzymes” using the transformed bone marrow-related cells of the present invention means decreasing the level of those enzymes in a hepatic disease to one third or less, preferably to one fifth or less, more preferably to one tenth or less, further more preferably to 1/20th or less, more preferably to 1/30th or less, and most preferably to a normal level. As described in Example 2 below, the transformed bone marrow-related cells of the present invention can decrease the level of GOT, GPT, and LDH in hepatic disease to about 1/23, 1/22, and 1/14, respectively. GOT, GPT, and LDH stand for glutamic-oxaloacetic transaminase, glutamic-pyruvic transaminase, and lactate dehydrogenase, respectively.

According to the present invention, the administration route for the transformed bone marrow-related cells of the present invention is not particularly limited as long as safe and efficient administration is possible. So long as these conditions are met, administration may be via peripheral blood vessels, subcutaneous, intramuscular, local, intraperitoneal, intraventricular, intraspinal, or intrathoracical. Administration via peripheral blood vessels or subcutaneous administration is preferable, and administration via peripheral blood vessels is more preferable. Those skilled in the art can select appropriate forms for the above administration route, without limitation. For example, by suspending the transformed bone marrow-related cells of the present invention in buffers and such, they may be used in the form of solutions. The concentration of transformed bone marrow-related cells used for administration is generally around 1×10⁴ cells/ml to 1×10¹¹ cells/ml of cell density per day when in solution form, and the administered volume is around 1 ml to 1000 ml. The amount for one day may be administered by division into an appropriate number of administrations. Those skilled in the art can appropriately adjust these doses according to a patient's age, body weight symptoms, administration route, administering institution, therapeutic process, and administration form. Animals to be the administration target are not particularly limited but include, for example, desirable mammals including humans and non-human mammals such as mice, rats, dogs, pigs, cats, cows, rabbits, sheep, goats, and monkeys.

To prevent blood clotting and embolus caused by contamination of transfusion solution with platelets, an antihistaric agent (hydroxyzine 25 mg, chlorphenirne 5 mg, or the like) or adrenocortical steroid (hydrocortisone 100 mg) may be administered before injection. When frozen cells are used for transplantation, if the hemolysis of erytrocytes in the cell suspension for transplantation becomes a problem, a haptoglobin preparation (4000 U) may be administered by drip infusion to prevent damage to the renal tubules. In case of peripheral blood stem cells, it is also preferable to inject cells in two days.

The present invention provides methods for preparing transformed bone marrow-related cells, comprising the step of using gene-carrying vectors to introduce genes into bone marrow-related cells taken from mammals. The methods for preparing the transformed bone marrow-related cells of the present invention comprises (i) collecting bone marrow-related cells, (ii) integrating a gene into a vector, and (iii) introducing the vector prepared in (ii) into the bone marrow-related cells in (i). The methods which can be used for each of the above steps are described above. Preparation of bone marrow-related cells and construction of the vector can be performed independently of the introduction of the vector into the bone marrow-related cells.

Vectors can be introduced into bone marrow-related cells by, for example, calcium phosphate methods (Graham F. L. and van der Eb, J. Virology, 1973:52; 456; Wigler, M. and Silverstein, S., Cell. 1977: 11; 223; Chen C. and Okayama H. Mol. Cell. Biol., 1987: 7; 2745), using a variety of transfection reagents, or by electroporation in case of plasmid vectors or such. The transfection reagents used may be DEAE-dextran (Sigma, # D-9885, M. W. 5×10⁵). DOTMA (Roche), Superfect (QIAGEN, #301305), DOTAP, DOPE, DOSPER (Roche, #1811169), TransIT-LTI (Mirus, Product No. MIR2300) and the like. When viral vectors are used, they can be introduced by contacting bone marrow-related cells with the viral vectors in appropriate physiological aqueous solutions. Bone marrow-related cells may be contacted with viral vectors inside (in vivo) or outside (in vitro or ex vivo) the body; for example, desirable physiological aqueous solutions such as culture media, physiological saline, blood, plasma, serum, and body fluids can be used.

The MOI (Multiplicity Of Infection; the number of infective viruses per cell) of the viral vectors contacted with the bone marrow-related cells is preferably one to 500, more preferably two to 300, more preferably three to 200, further more preferably five to 100, and more preferably seven to 70. Bone marrow-related cells introduced with vectors may be prepared as compositions for transplantation by combination with desirable pharmaceutically acceptable carriers or vehicles. A “pharmaceutically acceptable carrier or vehicle” means any solution in which living cells can be suspended, without limitation. For example, phosphate buffered saline (PBS). sodium chloride solution, Ringer's solution, culture medium, or such are included.

The present invention provides methods for using recombinant gene-carrying vectors to prepare the transformed bone marrow-related cells of the present invention.

The present invention also provides methods for maintaining and/or repairing tissues, comprising the step of administering the transformed bone marrow-related cells of the present invention into a living body.

The present invention further provides methods for diagnosing diseases using the transformed bone marrow-related cells of the present invention. The methods of the present invention may be performed by administering transformed bone marrow-related cells of the present invention introduced with a vector carrying a marker gene into the body, and observing the repair process in diseased tissues.

In an embodiment of the present invention, it is possible to use adexGFP, GFP-SeV/ΔF, and such, in which viral vectors are introduced with a gene encoding GFP as a marker gene. More specifically, when rats are used as described in Example 3 below, adexGFP or GFP-SeV/ΔF is introduced into collected bone marrow cells, and the transformed bone marrow cells are administered into the peripheral blood vessels of the rats. Detailed procedures for collecting bone marrow cells can be carried out according to the report by Terai et al. (2003) (as described above). At certain times after administration (for example, 24 hours after and 48 hours after), tissues (for example, liver or liver tissues) are collected from rats, freeze dried, and tissue sections are prepared. Such methods for collection and fixation of tissues, and preparation of sections can be performed according to the standard methods. The course of the tissue repair process may be observed by, for example, staining tissues collected after a certain time using direct or indirect immunofluorescence, and using a fluorescence microscope to examine the localization of the above bone marrow cells in the tissues. Tissue observation using fluorescent staining can be carried out according to the methods of Terai et al. (2003) and Li et al. (2000) (both described above). Those skilled in the art can use appropriate methods for tissue observation depending on the type of marker genes used.

### EXAMPLES

Herein below, the present invention will be specifically explained with reference to Examples, but it is not to be construed as being limited thereto.

Those skilled in the art can easily modify or change the present invention based on the descriptions of the present specification, and these are included within the technical scope of the present invention.

All references cited herein are incorporated as part of the present specification.

### Example 1

### Evaluation of Gene Expression

Gene expression in bone marrow cells was evaluated using an adenoviral vector carrying the GFP gene (adexGFP) and an F-gene-deleted Sendai virus vector carrying the GEP gene (GFP-SeV/ΔF). Bone marrow cells were collected from the thighbones of male Wister rats (10 to 12 weeks old; body weight: 250-300 grams/rat; CLEA Japan Inc.). Bone marrow cells were collected according to the method described by Terai et al. (described above). A suspension of prepared bone marrow cells was seeded into 24-well plates (Corning) at 1 ml per well. The cultured bone marrow cells were promptly infected with adexGFP or GFP-SeV/ΔF at 20 or 10 MOI, respectively. The cells were then stirred with a magnetic stirrer for 15 minutes, and then cultured at 37° C. for 30 minutes. This process was repeated once more. Then, the cells were washed twice with PBS, and physiological saline was added at 1 ml per well. 48 hours after gene introduction, bone marrow cells were harvested, and gene expression was evaluated using FACS (Becton. Dickinson and Company).

Gene expression was detected in 80% or more of the bone marrow cells, for both adexGFP and GFP-SeV/ΔF. The strength of gene expression (mean channel shift) was 12.6+0.2 for adexGFP and 16.6±0.4 for GFP-SeV/ΔF, indicating that the Sendai virus was superior. The strength of gene expression was 1.9±0.5 for non-infected cells in control groups.

### Example 2

### Repair of Liver Function in a Hepatic Disease Animal Model

The effect of the transformed bone marrow-related cells of the present invention on the repair of diseased tissue was examined using laboratory animals. Male Wister rats were used, as described in Example 1. As a hepatic disease model, an acute hepatic insufficiency model was prepared by intraperitoneally injecting a rat with carbon tetrachloride (CCl₄, Sigma) at 0.4 ml/kg/rat. Bone marrow cells were collected according to the method described by Terai et al. (described above). Bone marrow cells were collected from the thighbones of isogenic rats, and adjusted to 1×10⁸ cells/ml in a balanced salt solution. The gene to be introduced into the cells was an HGF- or hFGF-2 gene, and an adenoviral vector carrying the H1GF gene (adexHGF, RIKEN Gene Bank, RDB No. 1553), or the F-gene-deleted Sendal virus vector carrying the hFGF-2 gene (hFGF-2-SeV/ΔF; Li, O. H. et al. J. Virol. 74: 6564-6569 (2000); Masaki I. et al. Circ. Res. 90: 966-973 (2002)) was used as a recombinant viral vector.

The prepared cell suspension was seeded into 24-well plates (Corning) at 1 ml/ well. The cultured bone marrow cells were promptly infected with adexHGF or hFGF-2-SeV/ΔF at 20 or 10 MOI, respectively. The cells were then stirred with a magnetic stirrer for 15 minutes, and cultured at 37° C. for 30 minutes. This process was repeated once again. Then, the bone marrow cells were washed twice with PBS, and physiological saline was added at 1 ml/well to prepare a suspension of bone marrow cells for administration. Four hours after CCl₄ adminlstration, 1 ml of the prepared bone marrow cells (1×10⁸ cells/ml) was injected into tle rats via the peripheral vein (tail vein). At 24 hours after CCl₄ administration, sera were collected, and the level of serum liver enzymes (GOT, GPT, and LDHF) was measured. The enzyme activities of GOT, GPT, and LDH were respectively measured using CicaLiquid AST, CicaLiquid ALT, and CicaLiquid LDHJ kits (all from Kanto Chemical).

Table 1 shows the measured levels of serum liver enzymes (GOT, GPT, and LDH) 24 hours after CCl₄ administration in the rat acute hepatic insufficiency model. Normal values for each of GOT, GPT, and LDH are 30 IU/l or lower.

TABLE 1
Improvement in the level of serum liver enzymes in a hepatic
insufficiency model due to transformed bone marrow cells

| | GOT | GPT | LDH |
|---|---|---|---|
| Untreated group | 3,743 ± 855 | 2,656 ± 379 | 8,427 ± 1895 |
| Bone marrow cells only | 755 ± 321 | 618 ± 284 | 2,720 ± 630 |
| adexHGF-introduced | 482 ± 163 | 279 ± 94 | 1,327 ± 418 |
| bone marrow cells | | | |
| hFGF2-SeV/ΔF- | 163 ± 79 | 119 ± 81 | 584 ± 117 |
| introduced bone | | | |
| marrow cells | | | |

(Unit: IU/l)

Compared with the untreated group, there was a marked reduction in the levels of serum liver enzymes in groups administered with bone marrow cells introduced with the HGF or hFGF-2 gene. On comparison of HGF and hFGF-2, the latter had more profound effect. Compared to the untreated group, the GOT, GPT, and LDH levels of the hFGF-2-SeV/ΔF-introduced group were reduced to about 1/23, about 1/22, and about 1/14, respectively. These results indicate that over tle course of progressing liver damage, administration of gene-introduced bone marrow-related cells via the peripheral vein promotes the repair and treatment of liver damage.

### Example 3

### Observation of the Course of Tissue Repair

The course of liver tissue repair in the acute hepatic insufficiency rat model can be examined by administering bone marrow cells introduced with a marker gene such as GFP. Bone marrow cells transfected with adexGFP or GFP-SeV/ΔF were administered to rats via the peripheral blood vein, and then livers were removed from the rats after certain times, freeze-dried, and liver tissue sections were prepared. The course of the liver tissue repair 10 process can be observed by direct or indirect immnunofluorescent staining of the liver tissues, each collected after a certain time, then using fluorescence microscopy to examine the localization of the above bone marrow cells in the tissues. Tissue observation using fluorescent staining can be carried out according to the methods described in the report by Terai et al. (2003) and Li et al. (2000) (both described above).

### Example 4

### Therapeutic Effects of Gene-Introduced Bone Marrow Cells on Hepatic Cancer

A hepatic cancer model was prepared by intraperitoneally injecting male Wister rats (CLEA Japan Inc.) with 1% dimethylnitrosamine (DMN) once a week for seven weeks, beginning five weeks after birth, then farther feeding the animals with phenobarbital-containing food (Chubu Kagaku Shiryo) every day for 12 weeks. Six months after starting the experiment, the animals were used in the experiments described below. Bone marrow cells were transplanted according to the method described by Terai et al (Terai S. et al. J. Biochem. 134: 551-558 (2003)). Bone marrow cells were collected from the thighbones of the above Wister rats, and adjusted to 1×10⁸ cells/ml. Gene introduction was performed using an F-gene-deleted Sendai virus vector carrying a human interferon β (IFNβ) gene (hIMFβ-SeV/ΔF, Li H. O. et al. J. Virol. 74: 6564-6569 (2000)). Cells were infected in 24-well plates with hIFNβ-SeV/ΔF at MOI=10. 15 minutes of stirring with a magnetic stirrer and 30 minutes of incubation were repeated twice. The cells were then washed twice with PBS, and 1 ml of physiological saline was added to the cells. Bone marrow cells introduced with hIFNβ gene using hIFNβ-SeV/ΔF as prepared above (1×10⁸ cells) were injected into rats via the peripheral blood vein (tail vein). The animals were sacrificed one month after bone marrow cell injection, and the number of tumors and their longitudinal diameters were measured. The results were compared with those in control hepatic cancer model animals, which did not receive bone marrow cell injection.

As a result The number and diameter (mm) of tumors in the hepatic cancer model animals one month after bone marrow cell injection were 11±2.3 and 2.6±0.8 respectively for the untreated group (n=5); and 3.6±0.5 and 0.7±0.1 respectively for the group injected with bone marrow cells introduced with hIFNβ-SeV/ΔF (BM+hIFNβ-SeV/ΔF) (n=5) (mean±S.D.). These results indicate that administration of bone marrow cells that have been introduced with the hIFNβ gene using hIFNβ-SeV/ΔF suppresses the progression of hepatic cancers, or has an effect on the repair and treatment of hepatic cancers.

### Example 5

### Therapeutic Effects of Gene-Introduced Bone Marrow Cells in a Hepatectomy Model

A 70% partial hepatectomy (left hepatic lobectomy) was performed on male Wister rats (10 to 12 weeks old; 250 to 300 grams; CLEA Japan Inc.); and gene-introduced bone marrow cells were injected immediately after the operation. Bone marrow cells were transplanted according to the method described by Terai et al. (Terai S. et al. J. Biochem. 134: 551-558 (2003)). Bone marrow cells were collected from the thighbones of the above Wister rats, and adjusted to 1×10⁸ cells/mi. Gene introduction was performed using an F-gene-deleted Sendai virus vector carrying a gene encoding human FGF-2 gene (hFGF2-SeV/ΔF, Li H.O. et al. J. Virol. 71: 6564-6569 (2000); Masaki I. et at Circ. Res. 90 (9): 966-973 (2002)). Cells were infected in 24-well plates with the above vector at MOI=10. 15 minutes of stirring with a magnetic stirrer and 30 minutes of incubation were repeated twice. The cells were then washed twice with PBS, and 1 ml of physiological saline was added thereto. Immediately after the 70% hepatectomy (left hepatic lobectomy), the prepared bone marrow cells were injected into the peripheral blood vein (tail vein), sera were collected after 24 hours, and the level of serum liver enzymes was measured.

As a result. the GOT, GPT, and LDH levels 24 hours after the 70% hepatectomy and injection of bone marrow cells were 1186±137, 662±49, and 3250±205 respectively for the untreated group (n=5); and 764±82, 356±41, and 1266±110 respectively for BM+hFGF2-SeV/ΔF group (n=5) (mean±S.D.). These results indicate that during the progression of tissue damage in the liver following massive hepatectomy, it is possible to repair and treat the liver damage by administering bone marrow cells introduced with the hFGF2 gene using hFGF2-SeV/ΔF.

### INDUSTRIAL APPLICABILITY

According to the present invention, diseased tissues can be repaired using transformed bone marrow-related cells introduced with gene-carrying vectors. Diseases are not limited to inflammatory diseases. Regardless of immune diseases, cancers, genetic diseases, or such, the transformed bone marrow-related cells of the present invention can participate in repairing tissues with diseases or pathological conditions.

1. A transformed bone marrow-related cell introduced with a vector carrying a gene, wherein the cell is associated with the maintenance and/or repair of a tissue.

2. The transformed bone marrow-related cell of claim 1, wherein the gene is a marker gene, or has a function of directly participating in the maintenance and/or repair of a tissue, or of assisting a function of the transformed bone marrow-related cell in maintaining and/or repairing a tissue.

3. The transformed bone marrow-related cell of claim 2, wherein the gene with the function of directly participating in the maintenance and/or repair of a tissue, or of assisting a function of the transformed bone marrow-related cell in maintaining and/or repairing a tissue, encodes a protein or a peptide having an activity of controlling the differentiation or proliferation of a cell or of controlling a cellular function, wherein the protein or the peptide is selected from the group consisting of HGF, FGF, VEGF, PDGF, interleukin, GCSF, MCSF, SCF, IFN, Crx, and Otx2.

4. The transformed bone marrow-related cell of claim 1, wherein the vector is an adenoviral vector or a Sendai virus vector.

5. The transformed bone marrow-related cell of claim 4, wherein the adenoviral vector carries an HGF gene.

6. The transformed bone marrow-related cell of claim 4, wherein the Sendai virus vector carries an FGF2 gene.

7. The transformed bone marrow-related cell of claim 4, wherein the Sendai virus vector carries an IFN gene.

8. The transformed bone marrow-related cell of claim 1, wherein the bone marrow-related cell is a bone marrow cell or a bone marrow-derived cell.

9. The transformed bone marrow-related cell of claim 1, wherein the tissue is a diseased tissue.

10. The transformed bone marrow-related cell of claim 9, wherein the disease is a liver disease.

11. The transformed bone marrow-related cell of claim 10, which reduces a level of a serum liver enzyme.

12. The transformed bone marrow-related cell of claim 9, wherein the disease is a cancer.

13. The transformed bone marrow-related cell of claim 12, wherein the cancer is a hepatic cancer.

14. (canceled)

15. A method for preparing a transformed bone marrow-related cell, comprising the step of using a vector carrying a gene to introduce the gene to a bone marrow-related cell taken from a mammal.

16. (canceled)

17. A pharmaceutical agent for the maintenance and/or repair of a tissue, comprising the transformed bone marrow-related cell of claim 1 and a pharmaceutically acceptable medium.

18. The agent of claim 17, wherein the pharmaceutical agent is an agent for treating a liver disease.

19. The agent of claim 18, wherein the liver disease is a hepatopathy, hepatic insufficiency, cirrhosis, or hepatitis.

20-24. (canceled)

25. A method for manufacturing an agent for treating a liver disease, comprising the step of preparing a composition comprising the transformed bone marrow-related cell of claim 10 and a pharmaceutically acceptable medium.

26-31. (canceled)

32. A method of maintaining and/or repairing a tissue, comprising administering to a subject in need thereof transformed bone marrow-related cells introduced with a vector carrying a gene.

33. The method of claim 32, wherein the tissue is a diseased tissue.

34. The method of claim 33, wherein the disease is a liver disease.

35. The method of claim 33, wherein the disease is a cancer.

36. The method of claim 34, wherein the administration is a injection into a peripheral blood vessel of the subject.

37. The method of claim 32, wherein the gene is selected from the group consisting of HGF, FGF, FGF2, VEGF, PDGF, interleukin, GCSF, MCSF, SCF, IFN, Crx, and Otx2.

38. The method of claim 32, wherein the vector is an adenoviral vector or a minus-strand RNA viral vector.
